(19) United States
(12) Patent Application Publication
(10) Pub. No.: US 2005/0281768 A1
(43) Pub. Date: Dec. 22, 2005

(54) Method for washing curly or frizzy hair

(76) Inventor: Didier Saint-Leger, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/150,161

(22) Filed: Jun. 13, 2005

Related U.S. Application Data
(60) Provisional application No. 60/580,367, filed on Jun. 18, 2004.

(30) Foreign Application Priority Data
Jun. 11, 2004 (FR) 0451160

(51) Int. Cl. A61K 7/06; A61K 7/11

(52) U.S. Cl. 424/70.1

(57) ABSTRACT

The present disclosure relates to the use of a liquid detergent composition, with a viscosity ranging from 1 mPa/s to 100 mPa/s, comprising at least 5% of at least one detergent surfactant, for cleansing curly or frizzy hair and the scalp thereof, and a method for cleansing them using said liquid detergent composition.

This application claims benefit of U.S. Provisional Application No. 60/580,367, filed Jun. 18, 2004, the contents of which are incorporated herein by reference. This application also claims benefit of priority under 35 U.S.C. § 119 to French Patent Application No. 04 51160, filed Jun. 11, 2004, the contents of which are also incorporated herein by reference.

The present disclosure relates to the use of a liquid hair composition intended for cleaning curly and/or frizzy hair, and a method for cleansing the latter using this composition.

Hair tends to lose some of its qualities under the action of factors such as natural regreasing, sweat, elimination of scales, pollution, moisture, and the like. These factors can damage the visual appearance and the feel of the hair. For example, regreasing (and sometimes pollution) increase the weight of the hair, and such hair can tend to form into a mass. The hair may then become difficult to style, it may have an unpleasant greasy shine and can have a waxy feel which is also unpleasant.

The extent of the consequences of these factors, which are almost unavoidable, is variable. It depends, for example, on the quality of the hair, on its length and on the hairstyle adopted.

Whatever the case, to combat this inconvenience, use is made of shampoos, compositions based on detergent surfactants of various types, whose wetting effect allows the emulsification of the greasy compounds (such as the sebum) and their elimination during rinsing. These shampoos can generally make it possible to also eliminate dirt and dandruff.

In the great majority of cases, such formulas, although effective and pleasant, are of a thick or viscous physicochemical nature.

This thick consistency or high viscosity (for example ranging from 2,000 to 20,000 Centipoises, determined by a rotary Rheometer at the speed of 1 s⁻¹, 1, Centipoise being equal to 1 MilliPascal/s) is such that the products marketed are all in the form of a packaging of the flexible bottle type, the product flowing slowly to a greater or lesser degree by gravity, depending on its actual viscosity, or by forcing it by the pressure of the fingers on the flexible walls of the packaging.

On straight, curved or wavy hair, as may be found in Asian or Caucasian human groups, this high formula consistency does not at all prevent a fairly rapid initiation of the foaming shampoo composition and its easy spreading on the scalp and the hair. On the other hand, on highly curled or frizzy hair, such as that of populations of African ethnic groups, or their affiliated ethnic groups such as, for example, the African-American or Caribbean ethnic groups, this consistency can causes not only a particularly late and slow initiation of the foaming shampoo composition, but also cleansing of the scalp which may not be very effective through a lack of close contact between the thick washing base and the scalp.

A review of such specific problems of these human ethnic groups can be found in the book “Dermatologie sur Peau noire en France métropolitaine” [Dermatology on black skin in metropolitan France], by Drs Fitoussi and Sulimovic, Medécine-Sciences-Flammarion, 2003 or in the article “Ethnic hair updates: Past and Present”, A. J. McMichael, J. Am. Acad. Dermatol.; 48,127-133, 2003.

Accordingly, the present disclosure relates to a method of washing curly, for instance, ethnic hair, which can avoid at least one of the above problems.

For example, the present disclosure relates to a rapid method for cleansing hair which can lead to, at the end of the operation, hair having a clean feel and visual appearance.

It has now been found by the inventor, most unexpectedly and surprisingly, that this desired method, and others, could be achieved using liquid cosmetic compositions comprising, in a cosmetically acceptable medium, at least 5% of at least one detergent surfactant. This discovery forms the basis of the present disclosure.

The method using this composition as disclosed herein allows for the washing of the hair and of the scalp to be not only rapid but can prove to be complete, that is to say free of sebum, of a majority of the surface microbial flora, of dead cells and of residues from previous hair treatments.

Thus, the present disclosure relates to a method for cleansing curled, curly or frizzy hair of the corresponding scalp, comprising:
applying to wet or dry hair a liquid composition comprising, in a cosmetically acceptable medium, at least 5% of at least one detergent surfactant,
massaging the hair and the scalp, optionally with the aid of the fingers, and
rinsing the hair with water,
   wherein the viscosity of the composition ranges from 1 mPa/s to 100 mPa/s.

The present disclosure also relates to the use of a liquid detergent composition for cleansing curly or frizzy hair and the scalp.

The present disclosure still further relates to a method of obtaining good initiation of foam and/or a better reduction of the residual quantities of sebum and/or a better reduction of the resident flora on curled, curly or frizzy hair comprising applying the compositions disclosed herein, massaging the scalp, and rinsing the hair.

The viscosity of the compositions used for the method as disclosed herein can range, for example, from 1 to 100 Centipoises (mPa/s), for instance from 2 to 20 Centipoises (mPa/s), such as from 5 to 15 Centipoises (mPa/s).

The viscosities are measured at 25° C. with the aid of a rotary Rheometer at the speed of 1 s⁻¹.

The at least one detergent surfactant is chosen from anionic, amphoteric, nonionic and zwitterionic surfactants.

Thus, according to the present disclosure, the at least one detergent surfactant can be present, in a total amount of surfactant, ranging from 5% to 50% by weight, for instance from 6% to 30% by weight, such as from 8% to 25% by weight, of the total weight of the final composition.

The surfactants which are suitable for use according to the present disclosure include, for example, the following:

(i) Anionic Surfactant(s):

The nature of the anionic surfactants is not of critical importance within the context of the present disclosure.

Thus, by way of non-limiting examples of anionic surfactants that can be employed, by themselves or as mixtures, in the context of the present disclosure, there may be mentioned the salts (such as alkali metal, for instance sodium, salts, ammonium salts, amine salts, amino alcohol salts or magnesium salts) of the following compounds: alkyl sulphates, alkyl ether sulphates, alkylamido ether sulphates, alkyl aryl polyether sulphates, monoglyceride sulphates; alkyl sulphonates, alkyl phosphates, alkyl amide sulphonates, alkyl aryl sulphonates, α-olefinsulphonates, paraffin-sulphonates; alkyl sulphosuccinates, alkyl ether sulphosuccinates, alkyl amide sulphosuccinates; alkyl sulphosuccinamates; alkyl sulphoacetates; alkyl ether phosphates; acyl sarcosinates; acyl isethionates and N-acyltaurates, the alkyl or acyl radical of all these different compounds can comprise, for example, from 12 to 20 carbon atoms, and the aryl radical can denote for instance, a phenyl or benzyl group. Among the anionic surfactants which are also usable, further non-limiting mention may be made of the salts of fatty acids such as the salts of oleic, ricinoleic, palmitic and stearic acids, the acids of copra oil or of hydrogenated copra oil, and acyl lactylates in which the acyl radical comprises from 8 to 20 carbon atoms. It is also possible to employ weakly anionic surfactants, like alkyl-D-galactosideuronic acids and salts thereof, as well as the polyoxyalkylenated carboxylic ether acids and their salts, such as those comprising from 2 to 50 ethylene oxide groups and mixtures thereof. The anionic surfactants of the polyoxyalkylenated carboxylic ether acid or salt type can be, for example, those of formula (1):
R₁—(OC₂H₄)ₙ—OCH₂COOA (1)
wherein:
R₁ is chosen from alkyl, alkylamido and alkaryl groups, and n is an integer or a decimal number (average value) which can range from 2 to 24, such as from 3 to 10, wherein the alkyl radicals comprises from 6 to 20 carbon atoms, and, for instance, the aryl can be phenyl, A is chosen from a hydrogen atom, ammonium, Na, K, Li, Mg, or a monoethanolamine or triethanolamine residue. It is also possible to use mixtures of compounds of formula (1), for example mixtures in which the R₁ groups are different.

The compounds of formula (1) are sold, for example, by the company CHEM Y under the names AKYPO(NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50), or by the company SANDOZ under the names SANDOPAN (DTC Acid, DTC).

(ii) Nonionic Surfactant(s):

The nonionic surfactants themselves are also compounds which are well known per se (in this respect see for example the “Handbook of Surfactants” by M. R. Porter, published by Blackie & Son (Glasgow and London), 1991, pp. 116-178) and, in the context of the present disclosure, their nature is not of critical importance. They can thus be chosen from, by way of non-limiting example, polyethoxylated, polypropoxylated or polyglycerolated fatty alcohols, polyethoxylated, polypropoxylated or polyglycerolated fatty alpha-diols, polyethoxylated, polypropoxylated or polyglycerolated fatty alkylphenols or polyethoxylated, polypropoxylated or polyglycerolated fatty acids, all of which have a fatty chain comprising, for example, 8 to 18 carbon atoms, it being possible for the number of ethylene oxide or propylene oxide groups to range, for instance, from 2 to 50, and it being possible for the number of glycerol groups to range, for example, from 2 to 30.

The copolymers of ethylene oxide and propylene oxide and the condensates of ethylene oxide and propylene oxide with fatty alcohols may also be mentioned in a non-limiting manner; the polyethoxylated fatty amides can comprise, for example, from 2 to 30 mol of ethylene oxide, the polyglycerolated fatty amides on average comprising from 1 to 5 glycerol groups, such as from 1.5 to 4; the oxyethylenated fatty acid esters of sorbitan comprising from 2 to 30 mol of ethylene oxide; the fatty acid esters of sucrose, the fatty acid esters of polyethylene glycol, alkylpolyglycosides, the N-alkylglucamine derivatives, amine oxides such as the oxides of (C₁₀-C₁₄) alkylamines or the N-acylaminopropylmorpholine oxides. In one embodiment of the present disclosure, alkylpolyglycosides are used.

(iii) Amphoteric or Zwitterionic Surfactant(s):

The amphoteric or zwitterionic surfactants, the nature of which is not of critical importance in the context of the present disclosure, may be, by way of non-limiting example, derivatives of aliphatic secondary or tertiary amines in which the aliphatic radical is a linear or branched chain comprising from 8 to 18 carbon atoms and comprising at least one water-solubilizing anionic group (for example carboxylate, sulphonate, sulphate, phosphate or phosphonate); (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines may also be mentioned in a non-limiting manner.

Among the amine derivatives that may be used as disclosed herein, non-limiting mention may be made of the products sold under the name MIRANOL, as described in U.S. Pat. Nos. 2,528,378 and 2,781,354 and classified in the CTFA dictionary, 3rd edition, 1982, under the names Amphocarboxyglycinates and Amphocarboxypropionates of the respective structures:
R₂—CONHCH₂CH₂—N⁺(R₃)(R₄)(CH₂COO⁻) (2)
wherein:
R₂ is chosen from an alkyl radical of an acid R₂—COOH present in hydrolysed copra oil, and heptyl, nonyl and undecyl radicals,
R₃ is a beta-hydroxyethyl group and
R₄ is a carboxymethyl group; and
   R₂′—CONHCH₂CH₂—N(B)(C) (3)
   wherein:
B is chosen from —CH₂CH₂OX′ radicals,
C is chosen from —(CH₂)ₙ—Y′ radicals, with z=1 or 2,
X′ is chosen from a —CH₂CH₂—COOH group or a hydrogen atom,
Y′ is chosen from a —COOH group or the radical —CH₂—CHOH—SO₃H,
R₂′ is chosen from an alkyl radical of an acid R₂′—COOH present in copra oil or in hydrolysed linseed oil, and alkyl radicals, such as C₇, C₉, C₁₁ or C₁₃, a C₁₇ alkyl radical and its iso form or an unsaturated radical C₁₇.

By way of non-limiting example, there may be mentioned the cocoamphocarboxyglycinate sold under the trade name MIRANOL C2M concentrate by the company MIRANOL.

In one embodiment of the compositions for use in accordance with the present disclosure, at least one anionic surfactant is present.

In other embodiments, it is possible to use mixtures of surfactants, for example, mixtures of anionic surfactants, mixtures of anionic surfactants and of amphoteric and/or nonionic surfactants. In one embodiment of the present disclosure, a mixture comprising at least one anionic surfactant and at least one amphoteric surfactant is used.

The at least one anionic surfactant can be present in an amount ranging, for example, from 3% to 40% by weight, relative to the total weight of the cosmetic composition. For instance it can be present in an amount ranging from 5% to 25% by weight, such as from 8% to 20% by weight.

The at least one amphoteric and/or at least one nonionic surfactant, when present, can be present in an amount ranging from 0.5% to 20%, such as from 1% to 15%, relative to the total weight of the composition.

The cosmetically acceptable medium is a medium comprising water or water and at least one cosmetically acceptable organic solvent.

As used herein, the term organic solvent is understood to mean an organic compound which is liquid at a temperature of 25° C. and at atmospheric pressure. For example, the organic compound can be polar.

For further example, the solvent can be an alcohol. The alcohol can be chosen from, for instance, C₁-C₄ lower alcohols such as ethanol, isopropanol, tert-butanol, n-butanol; polyols such as propylene glycol, polyol ethers and mixtures thereof. In one embodiment of the present disclosure, the alcohol is ethanol.

In the cosmetically acceptable medium, water can be present in an amount ranging from 50% to 95% by weight, relative to the total weight of the composition, such as from 75% to 95%.

The organic solvents may be present in the composition in an amount ranging from 1% to 45% by weight, relative to the total weight of the composition.

The detergent cosmetic composition according to the present disclosure may additionally comprise at least one additive chosen from silicones in soluble, dispersed or microdispersed form; cationic surfactants; ceramides and pseudoceramides; soothing agents; antidandruff agents such as Climbazole, Piroctone Olamine, Zinc Pyridinethione, selenium sulphide, undecylenic acid and its salts; nonionic, cationic, amphoteric and anionic polymers and their salts, of the branched or unbranched random type; hair-structuring agents, chelating agents for minerals, reducing agents, oxidizing agents, vitamins and provitamins including panthenol; vegetable, animal, mineral and synthetic oils; waxes other than ceramides and pseudoceramides; water-soluble and fat-soluble sunscreens which are silicone-based or nonsilicone-based; glycerol; inorganic and organic pigments which are colored or noncolored; permanent or temporary dyes; pearlescent and opacifying agents; sequestering agents; plasticizers; solubilizing agents; acidifying agents; alkalinizing agents; antioxidants; hydroxy acids; penetrating agents; perfumes; perfume-solubilizing (peptizing) agents; preservatives; anticorrosion agents; rubefacient agents and treatment active agents such as agents against hair loss including Aminexil, and mixtures of these compounds.

Persons skilled in the art will be careful to choose the possible additives and their quantity so that they do not damage the properties of the compositions of the present disclosure.

These additives may be present in the cosmetic composition according to the present disclosure, when present, in an amount ranging from 0.000001% to 20% by weight, relative to the total weight of the cosmetic composition.

The composition may also further comprise at least one additive chosen from preservatives, pH-regulating agents and perfumes.

The pH of the compositions used according to the present disclosure can range from 2 and 12, such as from 3 to 8, for instance from 5 to 7.

The composition is dispensed over the hair and/or the scalp, for example in the form of droplets. The droplets can have, for instance an average volume ranging from 10 microlitres to 500 microlitres, such as from 50 microlitres to 200 microlitres.

The application of the composition may be carried out with the hands, with an applicator tip or with a dispensing comb. The application may also be carried out with the aid of a device which makes it possible to vaporize (or pulverize) the composition into the form of droplets. This may be, for example, a pump dispenser or an aerosol device.

The present disclosure also relates to a pump dispenser comprising a liquid cosmetic composition comprising at least 5% of at least one detergent surfactant.

The present disclosure still further relates to an aerosol device comprising at least one propellant and a liquid cosmetic composition comprising at least 5% of at least one detergent surfactant.

The present disclosure, of course, also relates to a method for washing curly or frizzy hair in which these devices are used.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

### Example 1

A composition A according to the present disclosure comprising 13% of Polyglyceryl-3-Hydroxylauryl ether, having a viscosity of 9 Centipoises (measured at 25° C. and at a shear rate of 1 s⁻¹), was compared with a thick, commercially available composition B, having a viscosity of 9500 Centipoises (neutralizing shampoo for sensitized hair from GOLDYS).

The time necessary for initiating the foam and for rinsing the composition were measured on a panel of models with the aid of a chronometer.

| | A | B |
|---|---|---|
| Initiation period | 2 to 10 seconds | 15 to 30 seconds |
| Rinsing period | About 1 minute | 2 to 3 minutes |

Study of Washed Scalp:

Two small zones of 1 cm² of scalp were made bare per half-head, by cutting the hair down to the scalp in order to have access to the said surface. On one of the two zones of each half-head, an apparatus for detection and quantification of sebaceous lipids such as the Sebumeter from the company Courage and Khazaka were used. On the other zone of each half-head, an adhesive strip such as D'Squame, sold by the company Cutec, was be applied so as to collect and then reveal and quantify the presence and the density of the Malassezia yeasts still present, according to the technique published by G. E. Piérard et al., in the article “Correlation between Malassezia Ovalis load and Dandruff severity”, J. Mycol. Med. 8, 83-86, 1998.

The combined implementation of these 2 determinations clearly showed a virtual absence of residual sebum in the zone washed with the composition A according to the present disclosure whereas sebum is present at high levels in the zone that was washed with the composition B of high viscosity. The same was true for the Malassezia colonization which droped markedly in the case where the composition according to the present disclosure, A, of low viscosity was used.

After drying, the hair washed with the composition according to the present disclosure wass cleaner, more aerated than that treated with the composition B. The feel was also more pleasant when the composition according to the present disclosure was used.

1. A method for cleansing curled, curly or frizzy hair and the corresponding scalp, comprising:
applying to wet or dry hair a liquid composition comprising, in a cosmetically acceptable medium, at least 5% of at least one detergent surfactant,
massaging the hair and the scalp, optionally with the aid of the fingers,
and rinsing the hair with water,
wherein the viscosity of the composition ranges from 1 mPa/s to 100 mPa/s.

2. The method according to claim 1, wherein viscosity of the composition ranges from 2 mPa/s to 20 mPa/s.

3. The method according to claim 1, wherein the at least one detergent surfactant is chosen from anionic, amphoteric, nonionic and zwitterionic surfactants.

4. The method according to claim 1, wherein the at least one detergent surfactant is chosen from anionic surfactants.

5. The method according to claim 1, wherein the at least one the detergent surfactant is chosen from mixtures of anionic surfactants and nonionic and/or amphoteric surfactants.

6. The method according to claim 1, wherein the at least one detergent surfactant is present in a total amount of detergent surfactant ranging from 5% to 50% by weight, relative to the total weight of the final composition.

7. The method according to claim 6, wherein the at least one detergent surfactant is present in a total amount of detergent surfactant ranging from 6% to 30% by weight, relative to the total weight of the final composition.

8. The method according to claim 7, wherein the at least one detergent surfactant is present in a total amount of detergent surfactant ranging from 8% to 25% by weight, relative to the total weight of the final composition.

9. The method according to claim 3, wherein the at least one anionic surfactant, when present, is present in a total amount for all anionic surfactants ranging from 3% to 40% by weight, relative to the total weight of the composition.

10. The method according to claim 9, wherein the at least one anionic surfactant, when present, is present in a total amount for all anionic surfactants ranging from 5% to 25% by weight, relative to the total weight of the composition.

11. The method according to claim 10, wherein the at least one anionic surfactant, when present, is present in a total amount for all anionic surfactants ranging from 8% to 20% by weight, relative to the total weight of the composition.

12. The method according to claim 1, wherein the at least one amphoteric and/or at least one nonionic surfactants, when present, are present in an amount ranging from 0.5% to 20% by weight, relative to the total weight of the composition.

13. The method according to claim 12, wherein the at least one amphoteric and/or at least one nonionic surfactants, when present, are present in an amount ranging from 1% to 15% by weight, relative to the total weight of the composition.

14. The method according to claim 1, wherein the composition further comprises at least one adjuvant chosen from silicones in soluble, dispersed or microdispersed form; cationic surfactants; ceramides and pseudoceramides; soothing agents; antidandruff agents; nonionic, cationic, amphoteric and anionic polymers and their salts, of the branched or unbranched random type; hair-structuring agents; chelating agents for minerals; reducing agents; oxidizing agents; vitamins and provitamins; vegetable, animal, mineral and synthetic oils; waxes other than ceramides and pseudoceramides; water-soluble and fat-soluble sunscreens which are silicone-based or nonsilicone-based; glycerol; inorganic and organic pigments which are colored or noncolored; permanent or temporary dyes; pearlescent and opacifying agents; sequestering agents; plasticizers; solubilizing agents; acidifying agents; alkalinizing agents; antioxidants; hydroxy acids; penetrating agents; perfumes; perfume-solubilizing (peptizing) agents; preservatives; anticorrosion agents; rubefacient agents; and treatment active agents; and mixtures of these compounds.

15. The method according to claim 14, wherein the at least one antidandruff agent is chosen from Climbazole, Piroctone Olamine, Zinc Pyridinethione, selenium sulphide, and, undecylenic acid and its salts.

16. The method according to claim 1, wherein the cosmetically acceptable medium is a medium consisting of water, or comprising water and at least one cosmetically acceptable organic solvent.

17. The method according to claim 1, wherein the composition is dispensed in the form of droplets.

18. A method of obtaining good initiation of foam and/or a better reduction of the residual quantities of sebum and/or a better reduction of the resident flora, on curled, curly or frizzy hair and the scalp thereof, comprising
applying to the curled, curly or frizzy hair a liquid detergent composition comprising, in a cosmetically acceptable medium, at least 5% of at least one detergent surfactant, and wherein the detergent composition has a viscosity ranging from 1 mPa/s to 100 mPa/s
massaging the hair and scalp, and
rinsing with water,
wherein the composition provides at least one property chosen from a good initiation of foam, a better reduction of the residual quantities of sebum on said curled, curly or frizzy hair, and a better reduction of the resident flora on said curled, curly or frizzy hair.

19. A device for vaporizing or pulverizing a composition into the form of droplets, wherein the composition comprises a liquid detergent composition having a viscosity ranging from 1 mPa/s to 100 mPa/s comprising, in a cosmetically acceptable medium, at least 5% of at least one detergent surfactant.

20. The device according to claim 19, wherein the device is a pump dispenser.

21. The device according to claim 19, wherein the device is an aerosol device.
